(19) United States
(12) Patent Application Publication
(10) Pub. No.: US 2007/0066950 A1
(43) Pub. Date: Mar. 22, 2007

(54) Disposable absorbent garment with elastic ears

(76) Inventor: Todd L. Nelson, Eau Claire, WI (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/232,446

(22) Filed: Sep. 21, 2005

(51) Int. Cl. A61F 13/15

(52) U.S. Cl. 604/389; 604/385.3

(57) ABSTRACT

An at least partially elastic ear for use on a disposable diaper is provided. The at least partially elastic ear is rectangular in shape, the rectangular shape formed by a top edge, a bottom edge, a distal edge between the top edge and bottom edge proximate to at least one fastener, and a proximal edge between the top edge and bottom edge distal to the at least one fastener. Further, a disposable absorbent garment, such as a diaper, is provided with a pair of rectangular at least partially elastic ears.

### FIELD OF THE INVENTION

The present invention relates to articles which absorb and/or contain bodily exudates, including disposable absorbent articles such as diapers, adult incontinence products, training pants, and the like. More particularly, the invention relates to elastic ears suitable for use on disposable absorbent articles.

### BACKGROUND OF THE INVENTION

Infants and incontinent adults are generally clothed in a disposable absorbent garment such as a diaper or diaper-like garment that is capable of absorbing and containing human waste products. Diapers are garments which are drawn up between the legs and fastened about the waist of the wearer and are well known. Such garments may be manufactured from reusable woven cloth materials and worn in conjunction with a fluid-impervious outer garment. In recent years, however, disposable diapers have come into widespread use and have gained a high degree of acceptance as a consumer product. Disposable diapers are designed to combine a fluid-impermeable or fluid impervious outer garment with a disposable absorbent inner garment, forming an integral structure that is intended to be discarded after a single use.

Known diapers have generally been constructed with an hourglass shape to minimize the material that comprises the area of the diaper worn in the crotch area of the wearer, yet will cover the front and rear of the wearer, and provide more material around the outer legs for increased leakage control. The hourglass shape also allows the front and rear portions of the diaper to overlap to completely encircle the waist of the wearer for greater coverage and leak protection. Fastening devices are generally included on the overlap areas of the front and rear portions of the diaper, to secure the diaper on the wearer. Each side of the rear waist portion of the diaper comprises a fastening device, and each fastening device can be secured to a side of the front portion of the diaper, respectively. Conventional fasteners have typically included such devices as adhesive tape tabs located on the rear waist portion of the diaper and a complimentary fastener, such as a taping panel, on the front waist portion of the diaper. Other conventional fasteners used with diapers include the use of a hook-and-loop material on the front and rear waist portions of the diaper, and belt-type systems.

The fastening devices on the rear waist portion of the diaper can be attached to the rear waist portion of the diaper body by way of flaps or ears. The ears can extend from the main diaper body at the rear waist, with the fastening devices joined to the edges of the ears or affixed on the body of the ears. When the rear waist portion of the diaper is joined to the front waist portion of the diaper, the ears form a part of the leg opening. The ears allow for a less bulky, more comfortable fit.

Many modifications to the shape and construction of diapers and diaper ears have been made in an attempt to improve leakage performance and comfort. One of these modifications includes providing elastic portions to the diaper ears and extending the lower edge of the ears toward the crotch portion of the diaper, typically in a concavely arcuate shape, to provide additional leg and hip coverage and snug, comfortable fit. U.S. Pat. No. 5,496,298 (Kuepper et al.) is an example of such a modification. However, the additional ear material can add bulk to the diaper and decrease the efficacy of the fastener, as well as increase manufacturing costs.

Hence, it is desirable to provide an improved disposable diaper, or similar disposable absorbent garment, that addresses some or all of the noted inadequacies of known disposable diapers.

### SUMMARY OF THE INVENTION

The present invention is directed to a disposable absorbent garment defined by a front portion, a rear portion, and a crotch portion joining the front and rear portions. The disposable diaper absorbent garment comprises a topsheet disposed over a backsheet, the topsheet and the backsheet each having a front portion and a rear portion, the front portion and the rear portion of the topsheet together with the front portion and the rear portion of the backsheet defining front and rear waist sections that together define a waist opening. The disposable diaper absorbent garment also includes a crotch section disposed between the front waist section and back waist section, and adjacent the front waist section and the rear waist section. The crotch section also defines a pair of elasticized leg openings. An absorbent core with side edges is disposed between a portion of the backsheet and the topsheet, the core being generally positioned in the crotch section of the garment. The disposable absorbent garment further includes a pair of at least partially elastic ears attached to the rear portion of the garment, each ear having at least one fastener for securing the garment to the wearer by releasably joining an at least partially elastic ear to a respective front waist portion. The at least partially elastic ears are rectangular to minimize the bunching of ear material around the wearer's legs while improving comfort and reducing manufacturing costs.

In another aspect, the present invention is directed to an elastic ear for use on a disposable absorbent garment. The ear comprises elastic material of rectangular shape and an elastic ear is affixed to each side section of the rear waist portion of the disposable absorbent garment. The elastic ear further comprises a fastener attached to the ear for securing a disposable absorbent garment to its wearer by releasably joining the elastic ear to a corresponding part of the front waist portion of the disposable absorbent garment.

### BRIEF DESCRIPTION OF THE DRAWINGS

The invention may be more completely understood in consideration of the following detailed description of various embodiments of the invention in connection with the accompanying drawings, in which:

FIG. 1 is a plan view of the disposable absorbent garment with elastic ears according to the present invention.

FIGS. 2A and 2B are plan views of the rear waist sections of prior art diapers.

FIGS. 3A and 3B are plan views of embodiments of the disposable absorbent garment with elastic ears according to the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE PREFERRED EMBODIMENTS

The present invention relates to disposable absorbent garments. The term “disposable absorbent garment” includes absorbent garments suitable for use by incontinent adults and toddlers, as well as by infants. Disposable absorbent garments are not intended to be laundered or reconditioned for reuse, but are to be discarded after use. Disposable absorbent garments can be placed against the body of a wearer to absorb and retain exudates from the wearer. While the following description is made in the context of a diaper, it should be understood that the present invention is also applicable to other disposable absorbent garments, such as adult incontinence garments and toddler training pants.

In one embodiment, the disposable diaper comprises a topsheet 14 disposed over a backsheet 12, the topsheet 14 and the backsheet 12 each having a front portion and a rear portion, the front portion and the rear portion of the topsheet 14 together with the front portion and the rear portion of the backsheet 12 defining front 18 and rear 20 waist sections that together define a waist opening. The disposable diaper 10 also includes a crotch section 26 connecting the front portion and the rear portion of the diaper 10. The crotch section 26 is disposed adjacent the front waist section 18, and defines a pair of elasticized leg openings. A core 16 comprising absorbent material is disposed between a portion of the backsheet 12 and the topsheet 14, in the crotch section 26. The disposable diaper 10 further includes a fluid acquisition member disposed beneath the topsheet 14 and in the crotch section 26, the fluid acquisition member having side edges and a front edge; the fluid acquisition member side edges terminating inboard of the absorbent core 16 side edges and the front edge terminating inboard of the front waist section 18. The fluid acquisition member, in combination with the absorbent core 16, form absorbency spaces that have a slow rate of absorption and that provide an absorbency reserve to the portions of the absorbent core 16 disposed directly underneath the fluid acquisition member. Elasticized barrier flaps 30, 32 are attached to the topsheet and are disposed inboard of the leg openings.

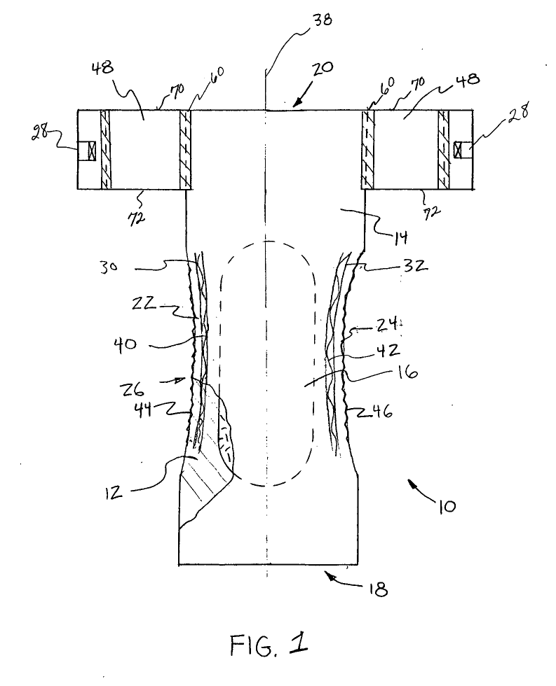

FIG. 1 is a plan view of a disposable diaper 10. Disposable diaper 10 typically comprises a backsheet 12, a topsheet 14 and an absorbent core or pad 16 disposed between backsheet 12 and topsheet 14. Topsheet 14 is made from a fluid pervious material and backsheet 12 is made from a fluid impervious material. The liquid impervious backsheet 12 can be composed of a thin plastic film that is flexible and can be elastic or inelastic. Alternatively, the backsheet 12 can comprise a laminate of a nonwoven sheet and a plastic film, such that the backsheet exterior is the nonwoven sheet, which provides for a diaper with an exterior surface that has a cloth-like texture. Suitable plastic films for use as the backsheet 12 liquid impervious layer generally include polypropylene films and polyethylene films. The topsheet 14 can be made of a woven or a nonwoven fabric, such that fluid can pass through the fabric and into the absorbent core 16. The topsheet 14 can be manufactured from materials such as synthetic fibers, natural fibers, or a combination of synthetic fibers and natural fibers. Materials for the topsheet 14 can include polyester, polyolefin or polypropylene fibers, cotton fibers, and wood/cellulose fibers. The absorbent core 16 can be manufactured from a number of liquid absorbent materials, including cellulose fluff, wood pulp fluff, superabsorbent materials, and combinations of superabsorbent materials and cellulose fluff.

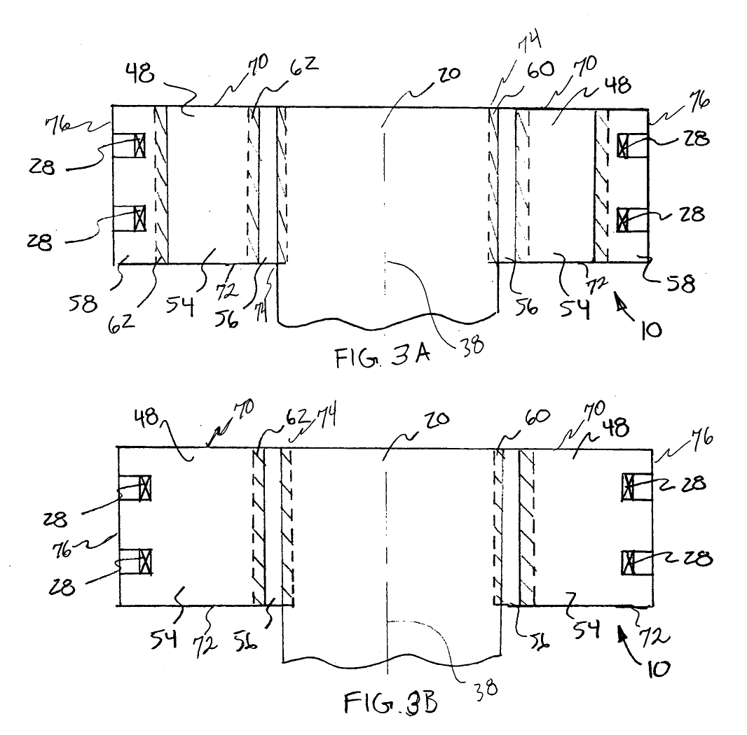

Topsheet 14 and backsheet 12 together form a shape with a rear waist section 20, a front waist section 18 and two leg portions 22, 24. Intermediate the front waist section 18 and rear waist section 20 is a crotch section 26. The disposable diaper 10 is placed around a wearer and held in place with fasteners 28, such as adhesive tapes, hook-and-loop material, or other connectors known in the art. The fasteners 28 can be attached to ears 48 extending from the rear waist portion of the diaper 10. When the fasteners 28 are affixed to the front waist section 18 of the diaper 10, the ears 48 form part of the leg opening, since the ears 48 are adapted, in use, to overlap with at least a portion of front waist section 18. The ears 48 may be extensions of the backsheet 12, topsheet 14, or combinations thereof. The ears 48 may also be non-integral to the backsheet 12, topsheet 14, or combinations thereof and joined at seam 60 to the rear waist section 20 of diaper 10. The joining at seam 60 of non-integral ears 48 to rear waist section 20 may be accomplished by adhesive bonds, sonic bonds, thermal bonds, stitching, or any of the various methods known in the art. As shown in FIGS. 3A and 3B, the present invention contemplates joining at seam 60 non-integral ears 48 to rear waist section 20 across the entire length of the boundary between the two pieces as well as only at select locations.

Further, a pair of barrier flaps 30, 32 is attached to topsheet 14 inboard of leg portions 22, 24 with a continuous bond. Alternatively, the barrier flaps 30, 32 can be attached to topsheet 14 by intermittent bonding. In one embodiment, where the barrier flaps 30, 32 are attached to topsheet 14, the barrier flaps 30, 32 are made from a different material than the topsheet 14. The preferred material for barrier flaps 30, 32 is one that is fluid pervious with pressure. Such a material blocks the passage of fluid therethrough under the pressures normally experienced in a disposable diaper but that allows fluid passage at pressure substantially higher.

Barrier flaps 30, 32 may include at least one elastic member, indicated at 40, 42 formed therein. As indicated in FIG. 1, elastic members 40, 42 are located at the inwardly directed edge of barrier flaps 30, 32. Diaper 10 also includes elastic members indicated at 44, 46 in leg portions 22, 24 of diaper 10.

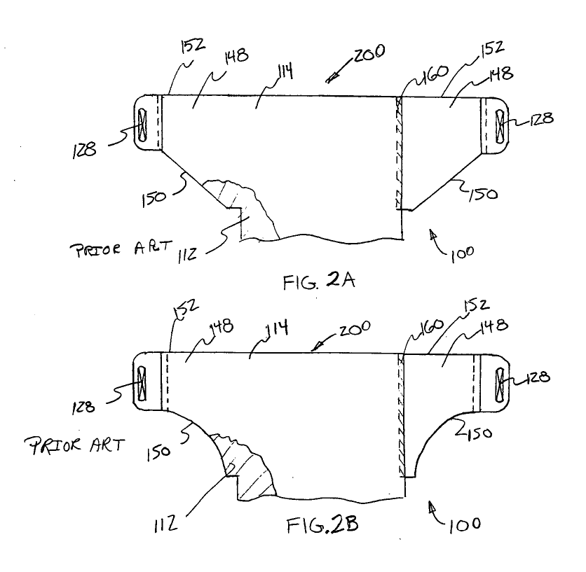

FIGS. 2A and 2B are plan views of the rear waist section 200 of two prior art disposable diapers 100. Ears 148 of prior art diaper 100 are non-rectangular in shape. FIG. 2A shows ears 148 with bottom edges 150 being generally linear and non-parallel to the top edges 152. FIG. 2B shows ears with bottom edges 150 that are non-parallel to top edges 152, but that are also non-linear. These prior art ears 148 claim the benefit of providing leg and hip coverage via ears 148 instead of overlapping front and rear portions as in previous diapers.

FIGS. 3A and 3B illustrate alternate embodiments of ears 48 of the present invention. The ears 48 have a proximal edge 74 adjacent the disposable diaper rear waist section 20, a distal edge 76, a top edge 70 and a bottom edge 72. The bottom edge 72 and the top edge 70 are linear and substantially parallel along their entire length. The distal edge 76 is linear and substantially parallel to the linear proximal edge 74. Alternatively, the distal edge 76 and the proximal edge may take on other than a linear shaped edge, such as a scalloped edge, a wavy edge and the like.

The ears 48 are rectangular in shape to minimize the amount of material located on the hip of the wearer. In addition, the rectangular shape is belt-like and secures the diaper about the waist of the wearer without significant downward stress on the ears and fasteners due to extended attachment of the ears to the body edge of the diaper. FIG. 3A shows a preferred embodiment of an ear 48 of a disposable diaper 10 constructed partially of elastic material. The ear 48 has an elastic portion 54 located between a proximate non-elastic portion 56 and distal non-elastic portion 58. At least one fastener 28 is attached to distal non-elastic portion 58 of each ear 48. Proximate non-elastic portion 56 acts as transition between elastic portion 54 and rear waist section 20 of diaper 10. Non-elastic portions 56, 58 may be made of non-elastomeric materials known in the art such as polymer films, woven fabrics, non-woven fabrics or the like. The choice of non-elastomeric materials is dependent on the desired properties of non-elastic portions 56, 58.

An alternative of the above-described preferred embodiment contemplates one non-elastic portion 56 or 58. The choice of whether to have a proximate non-elastic portion 56 or a distal non-elastic portion 58 is a matter of design choice. This alternate embodiment contemplates the same methods of joining at seam 62 elastic portion 54 to one of the non-elastic portions 56, 58 and joining at seam 60 ear 48 to rear waist section 20 of diaper 10.

FIG. 3B shows an embodiment of an ear 48 of a disposable diaper 10 constructed completely of elastic material. At least one fastener 28 is attached to elastic portion 54. Elastic portion 54 is joined at seam 60 to rear waist section 20 by any of the various methods described above.

Applicant has found that ears 48 of the preferred embodiment are constructed at least partially of elastic material. The elastic material can be formed from any type of elastic material capable of performing as described herein and include stretch-bonded laminate (SBL) materials, neck-bonded laminate (NBL) materials, elastomeric films, elastomeric foams, latent elastic materials and like combinations thereof. Elastic material may also be formed by combining non-elastic materials with any of the above elastic materials. Generally, the elastic material is capable of stretching in at least one direction. The elastic material may be capable of stretching in two directions. If the elastic material is capable of stretching in only one direction, the direction of stretch will be oriented so as to provide elastomeric forces in the direction from the fasteners 28 toward the centerline of the diaper 38.

The present invention may be embodied in other specific forms without departing from the essential attributes thereof; therefore, the illustrated embodiments should be considered in all respects as illustrative and not restrictive, reference being made to the appended claims rather than to the foregoing description to indicate the scope of the invention.

1. A disposable absorbent garment, comprising:
a main body comprising a backsheet, a topsheet disposed at least partially over the backsheet, and an absorbent core disposed at least partially between the backsheet and the topsheet, the main body defining a front waist section, a rear waist section, and a crotch section;
a pair of at least partially elastic ears wherein each of the at least partially elastic ears comprises a first non-elastic ear portion, wherein at least some, but less than all of the first non-elastic ear portion is joined to the rear waist section, and an elastic ear portion wherein the elastic ear portion is joined to the first non-elastic ear portion distal to the rear waist section, the at least partially elastic ears each defining a top edge and a bottom edge wherein the top edge and the bottom edge are substantially parallel along their entire lengths; and
at least one fastener attached to each of the at least partially elastic ears, distal to the rear waist section.

2. The disposable absorbent garment of claim 1 wherein the at least partially elastic ears are rectangular in shape.

3. The disposable absorbent garment of claim 2 wherein the at least partially elastic ears are elastic in at least one direction, the direction of elasticity being from the fastener toward the rear waist section.

4. (canceled)

5. (canceled)

6. The disposable absorbent garment of claim 1 wherein each of the at least partially elastic ears further comprises two or more non-elastic portions.

7. The disposable absorbent garment of claim 6 wherein a second non-elastic ear portion is joined to the elastic ear portion of each partially elastic ear distal to the rear waist section, and each of the at least one fasteners is attached to the second non-elastic ear portion.

8. The disposable absorbent garment of claim 7 wherein the at least partially elastic ears are elastic in at least one direction, the direction of elasticity being from the fastener toward the rear waist section.

9. The disposable absorbent garment of claim 2 further comprising a plurality of fasteners attached to at least one of the at least partially elastic ears distal to the rear waist section.

10. An at least partially elastic ear for use with a disposable absorbent garment comprising:
an elastic ear portion and a first non-elastic ear portion, the elastic ear portion joined to the first non-elastic ear portion and together defining a top edge and a bottom edge of the at least partially elastic ear wherein the top edge and the bottom edge are substantially parallel along their entire lengths; and
at least one fastener attached to the at least partially elastic ear.

11. The at least partially elastic ear of claim 10 wherein the at least partially elastic ear is rectangular in shape, the rectangular shape formed by the top edge, the bottom edge, a distal edge between the top edge and the bottom edge proximate to the at least one fastener, and a proximal edge between the top edge and bottom edge distal to the at least one fastener.

12. (canceled)

13. The at least partially elastic ear of claim 11 further comprising two or more non-elastic ear portions.

14. The at least partially elastic ear of claim 13 wherein the first non-elastic ear portion is located at the proximal edge of the at least partially elastic ear, a second non-elastic ear portion is located at the distal edge of the at least partially elastic ear, and further wherein the second non-elastic ear portion joins the at least one fastener to the at least partially elastic ear.

15. (canceled)

16. The disposable absorbent garment of claim 8 wherein the garment is a diaper.

17. The disposable absorbent garment of claim 14 wherein the garment is a diaper.

18. A disposable absorbent garment, comprising:
a) a main body comprising a backsheet, a topsheet disposed at least partially over the backsheet, and an absorbent core disposed at least partially between the backsheet and the topsheet, the main body defining a front waist section, a rear waist section, and a crotch section;
b) a pair of at least partially elastic ears wherein each of the at least partially elastic ears comprises
i) a first non-elastic ear portion, wherein at least some, but less than all, of the first non-elastic ear portion is joined to the rear waist section,
ii) an elastic ear portion, wherein the elastic ear portion is joined to the first non-elastic ear portion distal to the rear waist section,
iii) a second non-elastic ear portion joined to the elastic ear portion distal to the rear waist section,
the at least partially elastic ears each defining a top edge and a bottom edge wherein the top edge and the bottom edge are substantially parallel along their entire lengths; and
c) at least one fastener attached to each of the at least partially elastic ears, distal to the rear waist section.

19. The disposable absorbent garment of claim 18 wherein the disposable absorbent garment is a diaper.

20. The disposable absorbent garment of claim 18 wherein the fasteners are each attached to the second non-elastic ear portions.

21. The disposable absorbent garment of claim 18 wherein the at least partially elastic ears are elastic in at least one direction, the direction of elasticity being from the fastener toward the rear waist section.
